(19) United States
(12) Patent Application Publication
(10) Pub. No.: US 2009/0191603 A1
(43) Pub. Date: Jul. 30, 2009

(54) Use of rice bran as an accelerant in alcohol fermentation

(76) Inventors: Leo G. Gingras, Scottsdale, AZ (US); Paul R. Mathewson, Scottsdale, AZ (US)

Correspondence Address: MCDERMOTT, WILL & EMERY LLP;Attn: IP Department, 227 WEST MONROE STREET, SUITE 4400, CHICAGO, IL, 60606-5096, US

(21) Appl. No.: 12/321,678

(22) Filed: Jan. 23, 2009

Related U.S. Application Data
(60) Provisional application No. 61/023,803, filed on Jan. 25, 2008.

(51) Int. Cl. C12P 7/40; C12P 7/06; C12N 1/16

(52) U.S. Cl. 435/136; 435/161; 435/255.7

(57) ABSTRACT

The present invention relates to a method for production of fermentation-based products, through the fermentation of a carbohydrate substrate in the presence of a microorganism capable of fermentation. The fermentation process may be enhanced through use of a rice bran material as a nutrient source.

### CROSS-REFERENCE TO RELATED APPLICATIONS

This application claims priority to U.S. Provisional Application No. 61/023,803, filed Jan. 25, 2008, which is hereby incorporated by reference.

### FEDERALLY SPONSORED RESEARCH OR DEVELOPMENT

Not Applicable.

### TECHNICAL FIELD

This invention relates to the production of fermentation-based products, including ethanol, through the fermentation of a carbohydrate substrate in the presence of a microorganism. More specifically, this invention relates to the use of a rice bran as a fermentation accelerant in alcohol production.

### BACKGROUND OF THE INVENTION

The initial motivation for fuel ethanol production began in the mid-1970s as a result of the drive to develop alternative and renewable supplies of energy in response to the oil embargoes of 1973 (Calvin, M., 1974. Solar energy by photosynthesis. Science 184, 375-381; Calvin, M., 1980. Hydrocarbon from plants: Analytical methods and observations. Naturwissenschaften 67, 525-533). These initial efforts in developing biofuels did not gain progress at that time. With the easing of restrictions in foreign oil supplies, interest in biofuels gradually diminished. However, three decades later, there is a renewed interest in biofuel development (Somerville, C., 2006. The billion-ton biofuels vision. Science 312: 1277).

Current technology is capable producing two different types of liquid biofuels: bioethanol and biodiesel. Bioethanol and biodiesel account for 85% and 15% of current biofuel production respectively. Biodiesel is an alkyl ester of long chain fatty acids and is typically manufactured by means of an alkali, acid or lipase-catalyzed transesterification process of plant derived fat/oil with a short chain primary alcohol. Bioethanol is produced from plant-derived carbohydrates through microbial fermentation.

Corn-derived ethanol has been the main source of renewable biofuel in the United States. During 2007, 139 biorefineries in 21 states produced 7.8 billion gallons of ethanol making use of 22% of the total corn produced in the country. Expansion of the U.S. biofuel industry over the next 15 years may reduce dependence on foreign oil by 11.2 billion barrels per year accounting for $1.1 trillion and add $1.7 trillion (2008 dollars) to the U.S. economy during this fifteen years period.

It is estimated that corn-based ethanol production will reach 15 billion gallons per year in 2015 without interfering with the demand for human food and animal feed in the nation. However, to reach a target of 35 billion gallons of alternative fuels by 2017, there will be a need to exploit other feedstock for biofuel production. Efforts are being made to use agricultural wastes as feedstock for microbial fermentation leading to the production of bioethanol. Efforts are also being made to produce gasoline substitutes from plant-derived biomass materials through non-fermentative pathways involving a variety of microbial organisms.

Improvements in existing technologies as well as invention of new bioprocess technologies for the production of biodiesel, bioethanol or any other types of transportation fuel are urgently needed to meet the government mandates for biofuel utilization. The greatest challenge in the biofuel industry today is to produce biofuel at a price which is competitive with gasoline, but without government subsidies, and to realize enough capacity to meet the long term demand.

Fuel ethanol production represents one of the major industrial process involving microbial fermentation. Fuel ethanol production involves the fermentation of glucose leading to the production of ethanol. In the industrial production of ethanol, glucose is derived from starch. The corn is the traditional source of starch in fuel ethanol production. Starch is converted into simple sugars such as sucrose and glucose by amylase enzymes through a process referred as saccharification. Alternatively, the fuel alcohol, can also be produced through fermentation process involving sucrose derived from sugarcane. Currently fuel ethanol may be produced from corn starch or cane syrup utilizing either Saccharomyces cerevisiae or Zymomonas mobilis.

In a typical system for the production of ethanol, carbohydrate material derived from plant sources is subjected to a saccharification process to produce simple sugars. Simple sugars are then subjected to a microbial fermentation process to produce alcohol. The economic feasibility of producing fuel alcohol through a fermentation process depends greatly on the efficiency of the yeast-mediated conversion of sugars to alcohol. Any factor that increases the efficiency of the fermentation process, either in terms of speeding up the fermentation or producing a higher percentage of alcohol from the same amount of starting feedstock (or both), would greatly enhance the financial viability and value of the process.

Another approach to produce cost-effective bioethanol is to use less expensive reagents in bioethanol production. The present invention details a method of producing fermentation-based products including ethanol, using rice bran—a readily available by-product from the rice milling industry—to replace costlier sources of nitrogen and micronutrients necessary for microbial growth. In addition, the inventors of the present application have surprisingly discovered that the rice bran also acts as an accelerant in the alcohol fermentation process.

### SUMMARY OF THE INVENTION

The present invention provides a method for producing a fermentation-based product comprising the steps of providing a fermentation medium, a carbohydrate substrate, a micro-organism capable of fermenting the carbohydrate substrate and rice bran, and incubating the components for a time sufficient to produce a fermentation product.

In one embodiment, the present invention provides a method for accelerating the production of a fermentation-based product, including ethanol, comprising the steps of providing a fermentation medium, a carbohydrate substrate, a suitable strain of yeast, and rice bran.

In another embodiment of the present invention, stabilized rice bran is used as a source of nitrogen and micronutrients in a fermentation process. In yet another embodiment of the present invention, defatted rice bran is used as a source of nitrogen and micronutrients in a fermentation process.

In yet another embodiment of the present invention, the bran material is used as a source of nitrogen and micronutrients in microbial bioreactors used in the production of enzymes, therapeutic proteins, organic acids, antibiotics and other pharmaceutical compounds.

These and other aspects of the present invention will become more apparent when read with the following detailed description.

### BRIEF DESCRIPTION OF THE DRAWINGS

FIG. 1. Effect of addition of yeast extract (YE), raw rice bran (RB) and defatted rice bran (DRB) on ethanol production in a fermentation reaction involving baker's yeast and glucose. The yield of alcohol was monitored beginning from 16 hours after inoculation.

FIG. 2. Effect of addition of 1% yeast extract (YE), raw rice bran (RB) and defatted rice bran (DRB) on ethanol production in a fermentation reaction involving baker's yeast and glucose. The yield of alcohol was monitored from the beginning of inoculation.

FIG. 3. Effect of addition of 1.5% yeast extract (YE), raw rice bran (RB) and defatted rice bran (DRB) on ethanol production in a fermentation reaction involving baker's yeast and glucose. The yield of alcohol was monitored from the beginning of inoculation.

FIG. 4. Effect of addition of 2% yeast extract (YE), raw rice bran (RB) and defatted rice bran (DRB) on ethanol production in a fermentation reaction involving baker's yeast and glucose. The yield of alcohol was monitored from the beginning of inoculation.

### DETAILED DESCRIPTION

“Fermentation” or “fermentation process” refers to any fermentation process or any process comprising a fermentation step. A fermentation process includes without limitation, any process used to produce alcohols (e.g., ethanol, butanol); organic acids (e.g., citric acid, acetic acid, itaconic acid, lactic acid, gluconic acid); ketones (e.g., acetone); amino acids (e.g., glutamic acid); gases (e.g., H2 and CO2); antibiotics (e.g., penicillin and tetracycline); enzymes; vitamins (e.g., riboflavin, B12, beta carotene); and hormones. Fermentation processes also include fermentation processes used in the consumable alcohol industry (e.g., beer and wine), dairy industry (e.g., fermented dairy products), leather industry and tobacco industry. Such fermentation processes are well known in the art.

“Fermentation media” or “fermentation medium” refers to the environment in which the fermentation is carried out, including the substrate for fermentation. The substrate may be a simple sugar such as glucose, which is metabolized by the fermenting microorganism. Fermentation substrate may also be a complex carbohydrate which can be broken down either chemically or enzymatically to simple sugars suitable for fermentation by the microorganisms. Complex carbohydrate materials suitable for fermentation include starch derived form the endosperm of the cereal grains and other seed materials, as well as, the lignocellulosic materials derived from various plants. Fermentation media, including fermentation substrate and other raw materials may be processed through various means including milling, liquefaction and saccharification processes or other desired processes either prior to or simultaneously with the fermentation process.

Saccharification refers to the process by which complex carbohydrates are broken down into simple sugars which can act as the substrate for fermentation by the microorganisms. The saccharification and fermentation processes may occur in tandem in the sense that carbohydrate materials are broken down into simple sugars in the first stage followed by the second stage where the simple sugars are subjected to fermentation process. When the saccharification and fermentation processes are occurring simultaneously, it is referred as simultaneous saccharification and fermentation process (SSF).

It is known by those skilled in the art of using microbial cultures for the production of commercially valuable products, particularly through fermentation, that the growth of the microbial cultures, and in turn the yield of microbial products, can be enhanced by the addition of exogenous nutritive materials to the microbial growth medium. For example, yeast fermentation requires that live yeast have adequate nutrition in order to grow, multiply and ultimately consume the glucose substrate to produce alcohol. Glucose alone is typically not a complete source of nutrition for yeasts since glucose (or any other sugar) is composed of carbon and oxygen only. In addition to carbon and oxygen, yeast requires a source of nitrogen, metals and other nutrients in order to grow properly and metabolize the sugar into alcohol. In a typical fermentation process, yeast extract provides the additional nutrients.

Yeast extract is a commercially available exogenous nutritive source for a variety of microbial cultures. Yeast extract is a routinely used raw material in a variety of microbial culture media. Yeast extract is used both in fermentation and in non-fermentation media for microbial growth. Yeast extract is essential to improve the microbial growth rate and in improving the yield of microbial products in biotechnological applications. In addition to yeast extract, urea and ammonia are also used as the source of nitrogen in microbial growth media. Given its relatively expensive cost, it would be advantageous to replace yeast extract as a nutrient course.

The present invention relates to the use of bran material as a source of nitrogen in microbial culture media. Bran material, in addition to being a source of nitrogen, provides additional micronutrients, vitamins and phytosterols necessary for enhancing the growth of the microorganism, which increases the yield of microbial products. Since bran material is less expensive than other supplements, it has a definite economic advantage over other sources of nitrogen useful in microbial culture media.

In the present invention, the bran material used as a source of nitrogen and other micronutrients in microbial culture medium may be derived from a variety of cereal grains such as rice, wheat, oat, corn, rye, barley, sorghum, triticale, millet, buckweed, fonio, quinoa, teff, and kaniwa. Bran materials suitable for the present invention may also be derived from oil seeds such as sunflower, safflower, sesame, mustard, rapeseed, peanut, flax seed and soybean and the like. Preferably, the bran material useful in the present invention is derived from rice.

Cereal grains include three major portions namely, the endosperm, the germ, and the bran. The major portion of a cereal grain is made up of starchy endosperm. For example, in the case of wheat kernel, the endosperm accounts for about 80% weight percent while the bran/germ portion makes up approximately 20% weight percent of the grain. The term “bran” generally refers to the thin layer surrounding the endosperm in a cereal grain. In general, the bran fraction removed in the cereal milling operation contains some or all of the germ fraction of the cereal grain. As used in this invention, the terms “bran” or “bran fraction” includes some or all of the germ fraction. This mixed bran and germ fraction is also referred as “bran/germ” fraction. Modern cereal milling methods have the capacity to substantially remove the germ and bran portions from the endosperm portion. The bran and germ portions are considered by-products of the milling operation.

The current milling process for cereal grain has the ability to separate the bran and germ fractions in substantial quantity from the rest of the endosperm. The endosperm, hull and bran layer represent approximately 70%, 20%, and 10% respectively, of the rough rice. About 60 million metric tons of rice bran are generated from rice milling operation worldwide. However, this enormous amount of rice bran is not currently used in significant amounts, generally due to its instability. Yet, rice bran is a nutrient dense material derived from the milling of brown rice. The bran contains an array of nutritious components including protein, carbohydrate, oils and a significant quantity of micronutrients including vitamins, minerals and phytosterols that contribute positively to the metabolic processes of many organisms. These components are present naturally in the rice bran and are highly bioavailable.

In wheat milling, two different streams of products namely, the flour stream and bran/germ steam, are produced. The bran/germ portions may be separated into sub-categories generally referred to as “midds,” “shorts,” “germ,” “red dog'” and “bran.” Sometimes, the bran stream is sub-categorized as fine and coarse bran fractions. Any one stream of bran/germ fraction or any combination of different stream portions from the milling of grains can be used as a nutrient source in fermentation.

In corn milling, bran is derived from the pericarp located beneath the water impermeable cuticle. Because of its high fiber content, the pericarp is tough. In the corn milling operation, the corn is tempered by the addition of water and passed through a corn degerminator, which frees the bran and germ and breaks the endosperm into two or more pieces. The stock from degerminator is dried and passed through a separator and through a centrifugal-type aspirator to remove “aspirator bran.” The aspirator bran may contain some or all of the germ fraction.

The raw bran fraction derived from cereal grains has a higher lipid content along with significant lipolytic and oxidative enzyme activities. The milling process releases these enzymes, which can hydrolyze/oxidize the lipids associated with bran and germ fractions. Several methods have been developed to stabilize the germ and bran components of cereal grains. One of the methods used in the stabilization of cereal bran involves the application of direct heat (dry or wet heat) and mechanical extrusion. The mechanical extrusion process used for stabilizing the bran/germ fraction may further include addition of moisture to facilitate uniform heating of the bran/germ fraction. Stabilization by mechanical extrusion utilizes shear, friction, and pressure to generate the heat required to inactivate the lipolytic/oxidative enzymes. The stabilized bran material suitable for use as a nutrient source in the present invention can be obtained by using any one of the methods known in the art, including mechanical extrusion.

The bran/germ fraction can also be stabilized by extracting the oil using organic solvents to produce defatted bran (DFB). The defatted bran material can be produced from either the raw bran or the stabilized bran. Lipolytic enzymes associated with bran/germ fraction can also be inactivated using chemicals, such as hydrochloric acid, acetic acid, acrylonitrile, and proponal.

The efficiency of stabilization of bran by any of the known methods can be assessed by measuring the activities of the lipolytic and oxidative enzymes, such as lipase and peroxidases, before and after the stabilization process. One of the immediate effects of activation of lipolytic enzymes during the milling process is to release the fatty acids associated with the lipid component of the bran/germ fraction. Without a stabilization process, there is a steady increase in the accumulation of free fatty acid content of the bran/germ fraction. In the stabilized bran/germ fraction, the total fatty acid content in the bran/germ fraction should be less that 5% of the total extractable lipid content of the bran/germ fraction. Another measure for the efficiency of stabilization process is to monitor the microbial load. In the stabilized bran material, the total Coliform bacterial count is below 100 for one gram of the stabilized bran and Salmonella bacterium is undetectable. Similarly, yeast and mold show a maximum of 100 colony forming units per gram of the stabilized bran. Table 1 shows the typical chemical composition of stabilized rice bran and defatted rice barn useful in the present invention.

TABLE 1
Typical chemical composition of stabilized and
stabilized/defatted rice brans

| Analyte | Stabilized Rice Bran | Defatted Rice Bran |
|---|---|---|
| Energy (Cals) | 330.5 | 252 |
| Protein (g/100 g) | 14.5 | 17.6 |
| Total CHO (g/100 g) | 51 | 59.0 |
| Available CHO (g/100 g) | 22 | 34.9 |
| Fat (g/100 g) | 20.5 | 1.9 |
| Saturated Fat (g/100 g) | 3.7 | 0.5 |
| Total Sugars (g/100 g) | 8.09 | 7.1 |
| Ash (g/100 g) | 8 | 12.5 |
| Moisture (g/100 g) | 6 | 9.25 |
| Total dietary fiber (g/100 g) | 29 | 44.0 |
| B Vitamins (mg/100 g) | | |
| Thiamin-B1 | 2.7 | 3.24 |
| Riboflavin-B2 | 0.28 | 0.359 |
| Niacin-B3 | 46.9 | 41.33 |
| Pantothenic-B5 | 3.98 | 3.64 |
| Pyridoxal-B6 | 3.17 | 3.96 |
| B12 (mcg/100 g) | <0.5 | 1.15 |

The milling of oil seeds also produces a significant amount of bran material as a by-product. Bran materials derived from oil seeds can also be stabilized by similar methods useful in stabilizing the bran fraction derived from cereal grains. The stabilized oil seed bran is also suitable as a source of nitrogen and micronutrients in microbial culture medium of the present invention.

Raw bran, stabilized bran material, and defatted bran material can be used as a source of nitrogen and other micronutrients to replace currently used costlier nitrogen sources in the microbial culture medium. The microbial culture medium may be used to grow either a bacterial or a fungal species. The microbial culture medium may either be a fermentation medium or a non-fermentation medium.

“Fermenting microorganisms” refers to any microorganism suitable for use in a desired fermentation process. Suitable fermenting microorganisms according to the invention are able to ferment, i.e., convert, sugars, such as glucose or maltose, directly or indirectly into the desired fermentation product. Examples of fermenting microorganisms include a number of fungal organisms including yeast. Preferred yeast species include species of the Saccharomyces and in particular, Saccharomyces cerevisiae. Commercially available yeast suitable for fermentation process include, e.g., Red Star®/Lesaffre Ethanol Red (Available from Red Star/LeSaffre, USA) FALI (available from Fleischmann's Yeast, a division of AB Mauri Food, Inc., USA), GERT STRAND (available from Gert Strand AB, Sweden) and FERMIOL (available from DSM Food Specialties).

Microorganism useful in the present invention may also include non-fermentative microorganism. These non-fermenting microorganisms grows aerobically and are capable of using simple sugars as the source of carbon. These non-fermenting microorganisms can be genetically modified to have altered metabolic pathways to produce various types of organic compounds including high energy gasoline substitutes or the products which can be subjected to further processing to produce suitable transportation fuel. The non-fermenting microorganisms can also be used to produce a cost-effective commercial scale recombinant proteins with pharmaceutical application. The recombinant microorganisms can also be used to produce recombinant proteins such as enzymes suitable for industrial usage. The selection of microorganism suitable for expression of recombinant proteins depends on the requirement for secondary protein modifications. For example, unicellular eukaryotic yeast would be an ideal host for the expression of a recombinant protein with required glycosylation patterns. When secondary protein modification is not required, the protein expression can be carried out using E. coli as the host organism.

In addition to starch as the traditional carbohydrate source, another potential carbohydrate source for microbial production of ethanol is lignocellulose derived from plant sources. Lignocellulose contains about 35-50% cellulose, 20-35% hemicellulose, and 10-25% lignin. Cellulose is a linear polymer made up of glucose and is one of the most abundant carbonaceous materials available on earth; some billion tons of cellulose are being formed annually by the natural process of photosynthesis. Hemicellulose is a general term that includes all natural polysaccharides except cellulose and starch. Hemicellulose includes mixed polymers of xylose, arabinose, galactose, and mannose. Cellulose and hemicellulose polymers can be broken down into simple sugars with the help of cellulase and hemicellulase enzymes as well as by chemical means. Lignin is an aromatic polymer and is not a substrate for microbial fermentation.

Lignocellulosic feedstock may be selected from one or more of the following plants including switch grass, cord grass, rye grass, miscanthus, or a combination thereof. Lignocellulosic materials may also be derived from sugar cane bagasse, soybean stover, corn stover, rice straw, rice hulls, barley straw, corn cobs, wheat straw, oat hulls, corn fiber, wood fiber, or a combination thereof. Lignocellulosic feedstock may also comprise newsprint, cardboard, sawdust and combinations thereof. More preferably, lignocellulosic feedstock comprises oat hulls, wheat straw, switch grass, or a combination thereof.

The conversion of lignocellulosic materials into fuel alcohol requires the following steps: (1) cellulose and hemicellulose are liberated from lignin so that the cellulase enzyme gets an increased access to cellulose and hemicellulose; (2) cellulase and hemicellulase enzymes depolymerize the cellulose and hemicellulose molecules resulting in the release of free sugars; and, (3) fermentation of hexose sugars derived from cellulose and pentose sugars derived from hemicellulose to ethanol.

It is preferred that the lignocellulosic feedstock comprise a mechanically disrupted feedstock. Mechanical disruption of lignocellulosic feedstock may be performed according to any method known in the art capable of reducing the lignocellulosic feedstock into particles of an adequate size. For example, but not to be considered limiting, mechanical disruption of straw preferably results in pieces of straw having a length less than about 0.5 inches and an average diameter less than about 2 mm. Preferably, mechanical disruption of lignocellulosic feedstock produces particles which pass through about 20 mesh, preferably 40 mesh. Without wishing to be limiting, mechanical disruption of lignocellulosic feedstock may be performed by chopping, chipping, grinding, milling, shredding or the like. Preferably, mechanical disruption is performed by milling, for example, but not limited to, Szego milling, Hammer milling or Wiley milling.

Liberation of the cellulose and hemicellulose molecules from lignin is achieved by acid hydrolysis process involving the use of steam and acid. This acid hydrolysis process increases the accessibility of cellulose to cellulase enzyme. The term “cellulase enzymes,” or “cellulase,” refers to enzymes that catalyze the hydrolysis of cellulose to products such as glucose, cellobiose with two glucose molecules in β-1-4 linkage, and other oligosaccharides. Cellulase is a generic term denoting a multi enzyme mixtures comprising exo-cellobiohydrolases (CBH), endoglucanases (EG) and β-glucosidases (βG) that can be produced by a number of microorganisms. Commercially available cellulases suitable for use in the present invention include cellulases produced from fungi of the genera Aspergillus, Humicola, and Trichoderma, and from the bacteria of the genera Bacillus and Thermobifida. Cellulase produced by the filamentous fungi Trichoderma longibrachiatum comprises at least two cellobiohydrolase enzymes termed CBHI and CBHII and at least three EG enzymes. The processes of the present invention can be carried out with any type of cellulase enzymes, regardless of their source.

Optionally, the feedstock for fuel ethanol production using microbial fermentation may be derived from transgenic plants capable of breaking down the lignocellulosic materials post-harvest. For example, the transgenic plant may express cellulase enzymes derived from Thermomonospora fusca. Similarly in a transgenic plant, ligninase enzyme obtained from the white-rot fungus Phanerochaete chrysosporium can be expressed post-harvest. Depending on the degree of depolymerization of the cellulose and hemicellulose materials from the transgenic plants, appropriate microorganisms can be used to achieve the maximum conversion of the transgenic feedstock into fuel alcohol.

In the simultaneous saccharification and fermentation process, the lignocellulosic material is comminuted to a fine particle size (preferably less than 100 microns) and exposed to mixed culture of cellulolytic organism and a thermophilic ethanol-producing bacillus at appropriate temperature and pH. The comminutation can be carried out by conventional techniques such as by mechanical grinding or pulverizing the lignocellulosic material. Alternatively, to break down the cellulose component into simple sugars, the lignocellulosic material can be treated first with acid or alkali which penetrate the lignin and degrade or depolymerize the lignins sufficiently to make the cellulose available for contact with the cellulase enzyme produced by the cellulolytic organisms in the mixed culture.

Ideally, commercial production of ethanol from cellulosic biomass would employ a natural organism or a genetically modified organism capable of fermenting the monosaccharides such as glucose, galactose, mannose, xylose, arbinose and the disaccharide cellobiose produced from enzymatic digestion of cellulose. Preferably, such an organism would be able to hydrolyze resilient polymers such as cellulose or hemicellulose without the addition of exogenous enzymes or chemicals to break down the cellulose and hemicellulose molecules into simple sugars. These organisms would have the capacity to produce and secrete hydrolytic enzymes necessary for the breakdown of complex polymers of cellulose and hemicellulose into fermentable sugars.

It is possible to genetically manipulate the microorganisms to make use of cellulose or cellulose derivatives as the source of carbon for the production of ethanol through fermentation. A microorganism capable of expressing enzymes for breaking down the cellulose or cellulose derivatives can be genetically created. For example, an organism transformed with a plasmid carrying a gene coding for β-glucosidase may have the capacity to produce glucose from cellobiose derived from cellulose by an endo- or exocellular cellulase. In the same way, a microorganism capable of expressing and secreting cellulase may also be created. A microorganism containing both the cellulase and β-glucosidase genes would have the capacity to produce simple sugars from cellulose material. The ability to degrade cellulose or cellulose derivatives can be added to an ethanolgenic microorganism, which already has the capacity for carrying out the fermentation using simple sugar. Such a genetically manipulated microorganism may have the capacity to carry out simultaneous saccharification and fermentation. Ideally, the microorganism capable of carrying out simultaneous saccharification and fermentation is also ethanol tolerant to make the process of ethanol production form cellulosic feedstock more efficient.

Microorganisms may also be genetically manipulated to make use of all of the pentose sugars derived from hemicellulose component of the lignocellulosic materials. Xylose, a pentose sugar derived from break down of the hemicellulose may be converted into metabolizable xylulose sugar. The ability to convert the xylose into xylulose may be conferred to a microorganism by means of transforming it with a plasmid containing the gene coding for xylose isomerase. A yeast strain such as Schizosaccharomyces pombe ATCC No. 2476 with the exogenously added xylose isomerase gene may have the capacity for producing ethanol using both glucose and xylose.

Microbial fermentation may also be used to produce butanol for use as a fuel additive. Recombinant microorganisms expressing a 1-butanol biosynthetic pathway, a 2-butanol biosynthetic pathway and an isobutanol biosynthetic pathway are known in the art. Microorganisms that are tolerant to butanol have also been isolated by chemical mutagenesis. Higher-chain alcohols have energy densities close to gasoline, are not as volatile or corrosive as ethanol, and do not readily absorb water. Furthermore, branched-chain alcohols, such as isobutanol, have higher-octane numbers, resulting in less knocking in engines. Butanol may be produced using a batch method of fermentation or by continuous fermentation methods. A classical batch fermentation is a closed system where the composition of the medium is set at the beginning of the fermentation and not subject to artificial alterations during the fermentation. Continuous fermentation is an open system where a defined fermentation medium is added continuously to a bioreactor and an equal amount of conditioned media is removed simultaneously for processing.

It may also be possible to reengineer microorganisms to turn agricultural products into ready-to-use transportation fuels (Service, R. F. 2008. Eyeing oil, synthetic biologist mine microbes for block gold. Science 322, 522-523). Metabolic engineering may be used to modify the highly active amino acid pathway in Escherichia coli bacteria to produce isobutanol (Atsumi, S., Hani, T. & Liao, J. C. (2008). Non-fermentative pathways for synthesis of branched-chain higher alcohols as biofuels. Nature 451, 86-89.)

Microorganisms are also used in the waste management technologies such as syngas fermentation and domestic waste water treatment. Syngas fermentation is a microbial process. In this process, syngas, a mixture of hydrogen and carbon monoxide, is used as a substrate for microbial fermentation in a bioreactor. The main product of syngas fermentation is ethanol. The bran material of the present invention may be used as a source of micronutrients in accelerating the growth and metabolism of the microorganisms used in the syngas fermentation, and in the domestic waste water treatment.

Irrespective of the process used or the end products collected, microorganisms can be effectively and efficiently grown using bran material as the source of nitrogen and micronutrients in the absence of any other exogenous nitrogen source. In the case of biofuel production, by using appropriate amounts of bran material, the fermentation process can be adjusted to reach an optimum range for alternative fuel production. Bran material is also useful in enhancing fermentation rates, increasing in the overall efficiency of the fermentation process.

The above description is not intended to limit the claimed invention in any manner. The present invention is further illustrated in the following examples. However, it is to be understood that these examples are for illustrative purposes only, and should not be used to limit the scope of the present invention in any manner.

### EXAMPLE 1

### Evaluation of Rice Bran (RB) and De-Fatted Rice Bran (DRB) as a Source of Nitrogen and Micronutrients in Ethanol Production

This example illustrates the use of rice bran as the source of nitrogen and other micronutrients in a fermentation process for producing ethanol using Saccharomyces cerevisiae (bakers yeast). As shown in this example, rice bran may be substituted for yeast extract as a source of nitrogen and micronutrients in alcohol fermentation and to enhance the production of ethanol. Thus, the use of rice bran not only reduced the cost of ethanol fermentation, but also enhanced the ethanol production.

Table 2 shows the composition of the Medium 1 containing yeast extract (YE) as the nitrogen source. Table 3 shows the composition of Medium 2 containing raw rice bran (RB) in varying amounts. Table 4 shows the composition of Medium 3 containing defatted rice bran (DRB) in varying amounts. Table 5 shows the composition of control medium used to evaluate the production of ethanol in the absence of YE or RB or DRB. The components in the Tables 2-5 are expressed in % (weight/volume).

TABLE 2
Composition of culture medium containing yeast extract (YE)

| Component | Proportion (%) |
|---|---|
| Glucose | 2.00 |
| Ammonium Sulfate | 0.30 |
| K2HPO4 | 0.05 |
| ZnSO4 | 0.005 |
| MgSO4 | 0.005 |
| YE | Varied 1, 1.5, 2.0 |

TABLE 3
Composition of culture medium containing rice bran (RB)

| Component | Proportion (%) |
|---|---|
| Glucose | 2.00 |
| Ammonium Sulfate | 0.30 |
| K2HPO4 | 0.05 |
| ZnSO4 | 0.005 |
| MgSO4 | 0.005 |
| RB | Varied 1, 1.5, 2.0 |

TABLE 4
Composition of culture medium containing defatted rice bran (DRB)

| Component | Proportion (%) |
|---|---|
| Glucose | 2.00 |
| Ammonium Sulfate | 0.30 |
| K2HPO4 | 0.05 |
| ZnSO4 | 0.005 |
| MgSO4 | 0.005 |
| DRB | Varied 1, 1.5, 2.0 |

TABLE 5
Composition of control culture medium

| Component | Proportion (%) |
|---|---|
| Glucose | 2.00 |
| Ammonium Sulfate | 0.30 |
| K₂HPO₄ | 0.05 |
| ZnSO₄ | 0.005 |
| MgSO₄ | 0.005 |

Approximately 50 ml of medium was placed in a 250 ml conical flask and 1% baker's yeast (500 μl per flask) grown in yeast mold broth for 48 h was inoculated. The flask mouth was covered with thick parafilm to avoid the escape of ethanol produced. Flasks were incubated at 30° C. for 90 h at 100 rpm. First samples were drawn approximately at 14 h and subsequent samples were collected at an interval of 12-14 h. From each flask, 500 μl of sample was drawn to a 1.5 ml eppendoff tube, and the flasks were again covered tightly with parafilm and incubated on shaker. To the 500 μl of sample, 500 μl of water was added and vortexed briefly and centrifuged at 10,000 rpm for 5 min. After this, samples were filtered into HPLC sample vials using 0.45μ filters and stored for HPLC analysis. The HPLC analysis was carried out using Rezex-ROA Organic acid H⁺8% column. The flow rate was maintained at 0.6 ml/min and detection was done using a refractive index detector. The run time for a sample was 25 minutes. Absolute ethanol was used to prepare a standard with known concentrations. Calculation of ethanol content in the experimental samples was calculated using the following formula: Ethanol (mg/g)=(Sample peak area)/(Standard peak area)×(Concentration of standard)×(Dilution of sample). The ethanol content is expressed in % grams of ethanol produced per 100 g of broth.

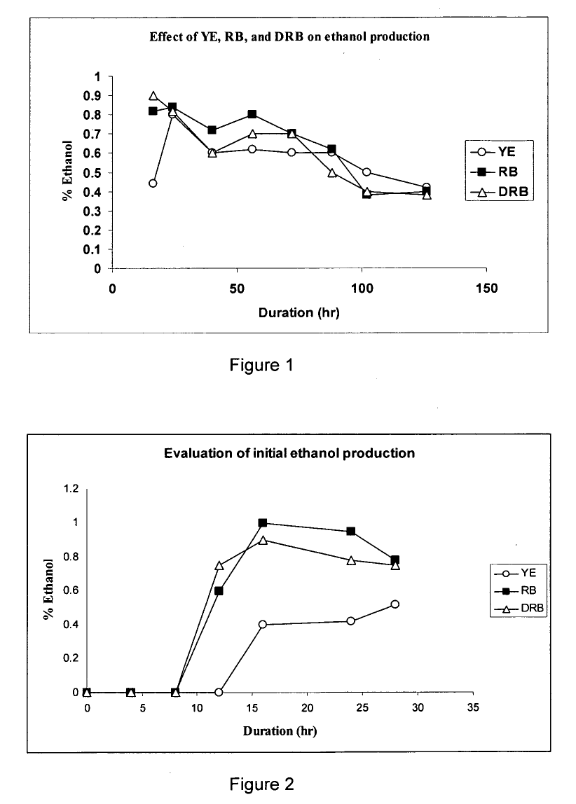

The first sampling was done 16 h after inoculation and subsequent sampling was done every 12 h interval. It is clear from FIG. 1 that in the samples containing either RB or DRB as the source of nitrogen, the maximum amount of ethanol was produced at 16 h and declined subsequently. From FIG. 1 it is also clear that ethanol production was improved when DRB or RB was used as a nutrient source when compared to the sample with YE as a nutrient source. In flasks that had no YE or RB or DRB, there was no yeast growth and ultimately there was no ethanol production.

Since the first sampling was done at 16 h, which is the point of maximum ethanol production, in the next set of experiments, sample were collected every 4 h from the time of inoculation. All the other conditions were kept constant. Shown in FIG. 2 is the profile of the ethanol production during the first 28 hours with 1% of YE or RB or DRB. It is clear from FIG. 2, that for the first 12 h, the YE based medium did not produce any ethanol. In the other two samples with RB or DRB as the source of nitrogen, the ethanol production peaked at about 16 hours, confirming the results of the previous experiment.

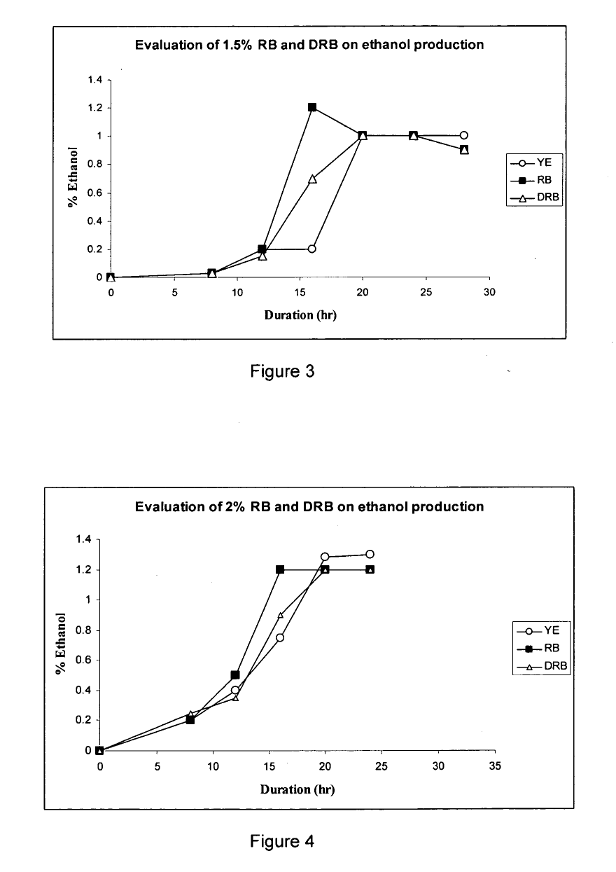

The experiment described in FIG. 2 was repeated using 1.5% YE or RB or DRB as the nutrient source. FIG. 3 shows the profile for ethanol production with 1.5% YE or RB or DRB as the source of nitrogen. Maximum ethanol concentration was attained at 16 hours for RB containing samples and at 20 hours for DRB and YE containing sample. From the profile, it is evident that fermentation improved when the concentration of YE as nutrient was increased from 1 to 1.5%. However, RB as a nutrient source was still more efficient and produced 1.2% ethanol at 16 hours, while the YE containing sample produced only 0.2% ethanol. The DRB containing sample was slightly less efficient than RB containing sample at 1.5% concentration.

The ethanol fermentation experiment was also performed using YE or RB or DRB at 2% nutrient concentration. The ethanol production profile for this experiment is shown in FIG. 4. Again, at 16 hours of fermentation, RB as a nutrient was better and attained the maximum ethanol concentration of 1.2%. Increasing the RB from 1.5% to 2% has had no effect on final ethanol concentration and on the profile. There was a significant improvement in the ethanol production when the concentration of YE was increased to 2%. However, the kinetics of ethanol production was still slower when compared to the sample with RB as the source of nitrogen.

Mean ethanol production in medium containing glucose was less than 1.0% due to limited amount of glucose (2.0%). This medium was designed to evaluate the performance of RB, DRB and YE as a source of nitrogen and micronutrients on ethanol production, and not to maximize the ethanol concentration and yield. Also, no antibiotics were added to prevent contamination of the ethanol fermentation. As the concentration of nutrient source increased in the fermentation medium, ethanol production also increased significantly. Maximum ethanol production occurred at the 2.0% dose level. Addition of a 2% concentration of any nitrogen source produced significantly greater ethanol than the samples containing a nitrogen source at either 1.5% or 1% level. Ethanol production does not vary significantly when a nitrogen source was used at 1% or 1.5% concentration.

Ethanol production with RB or DRB as the source of nitrogen were not significantly different from each other. The sample with YE as the nitrogen source produced significantly lower ethanol when compared to the sample with RB as the nitrogen source, but is similar to the sample with DRB as the nitrogen source. Ethanol production at time 8 h and 12 h were different significantly from all other time points. Ethanol production at time 16 h, 20 h and 24 h were significantly different from 8 h and 12 h. Ethanol production appears to peak at between 20 h and 24 h. Therefore the fermentation can be terminated at 20 h when using 2% initial glucose concentration.

From these experiments, it is clear that both raw rice bran and defatted rice bran are excellent sources of nitrogen, showing improvement over the use of yeast extract at all concentrations used. Increasing the nutrient concentration from 1.5% to 2% had a marginal effect for RB since the maximum ethanol concentration was already attained at 1.5% concentration. DRB as nutrient was also better than YE, but slightly less efficient when compared with RB.

### EXAMPLE 2

### Ethanol Fermentation with Corn-Derived Glucose Using RB and DRB as Nutrient Source at Shake Flask Level

Approximately 50 g of corn grain sample was ground in an Udy sample grinder and sieved through 850 μm and 600 μm mesh size. Fractions were collected separately and stored in poly bags.

Sample preparation for ethanol production was done according to Zhan et al. (2006, Ethanol production from supercritical-fluid-extrusion cooked sorghum. Industrial Crops and Products 23: 304-310). About 3 g of ground sample was placed in a 250 ml conical flask to which 24 μl of α-amylase (Liquizyme) and 10 ml water were added. Samples were incubated in a water bath at 85° C. for 45min. Later, a second dose of 8 μl of α-amylase was added and the temperature of water bath was lowered to 80° C. Samples were incubated for 30 min. After incubation, the samples were cooled to room temperature and 320 μl of glucoamylase (Spirozyme) was added. Samples were incubated for 2 h at 40° C. and additional 40 ml of water was added to the flasks. 500 μl of sample from each flask was collected and centrifuged at 5000 rpm for 10 min and supernatant was aspirated to an eppendoff tube and appropriate dilutions were made. Samples were filtered and quantified for glucose.

After corn grain samples were digested with enzymes to release glucose, raw rice bran (RB), defatted rice bran (DRB) and yeast extract (YE) were added at 1.0%, 1.5% and 2.0% to separate flasks. To the control sample, no nutrient was added. Duplicate samples were maintained for each concentration to help statistical analysis. In order to start the fermentation process, 1 ml of 48 hour-old bakers yeast (Saccharomyces cerevisiae) culture was added to each flask. Flasks were covered with paraffin to avoid escape of ethanol produced. About 100μ of sample was drawn at 8, 12, 16, 20, 24 h intervals from each flask and used for ethanol estimation using HPLC. Samples were also drawn at specific time points and were analyzed for glucose utilization after appropriate dilution. To control contamination, antibiotic lactrol was used at the final concentration of 2 PPM.

Glucose and ethanol levels were estimated using the method described in Ko et. al. (2005 Simultaneous Quantitative Determination of Monosaccharides Including Fructose in Hydrolysates of Yogurt and Orange Juice Products by Derivatization of Monosaccharides with p-Aminobenzoic Acid Ethyl Ester Followed by HPLC Bull. Korean Chem. Soc., 26(10) 1533-1538). This method was slightly modified to detect glucose and ethanol simultaneously. High performance liquid chromatograph (HPLC) equipped with refractory index detector (RID) was used in the current study. Water was used as mobile phase with a flow rate of 0.6 ml/min. Rezex-Organic acid column was used for separation and quantification of glucose and ethanol. Data were acquired using Lab solutions software and statistically analyzed using SAS software package.

In these experiments, corn grain was enzymatically digested to sugars using the procedures described above. The sugar released was then fermented to ethanol at shake flask level. Experiments were performed using RB, DRB and YE as nutrient sources at 1%, 1.5% and 2% concentrations along with control where no nutrient was added. The fermentation profiles of glucose utilization and ethanol production at different nutrient concentrations and at different time points (0, 4, 8, 12, and 24 hours) are shown in Table 6. It is evident from the Table 6, that glucose utilization rates were fastest with the addition of yeast extract and increasing the YE concentration from 1% to 2% did not enhance the glucose utilization at 8 hours of fermentation. Glucose utilization rates for RB and DRB were lower than with YE, while the rates of glucose utilization were enhanced with increasing the RB and DRB nutrient concentration from 1% to 2%. The control experiment, which contained no nutrients, exhibited slowest glucose utilization rates until 12 hours. Accordingly, ethanol production followed the same trend as glucose utilization. YE enabled higher ethanol concentration by 8 hours compared to other nutrients and control. At 12 hours, ethanol concentrations converged to almost similar values for all three groups with added nitrogen source except the control group with no exogenous nitrogen source. At 24 hours, the final ethanol concentration and glucose utilization were similar for all samples, including the control, which suggests that the addition of nutrients affects the rates and not the final concentration of ethanol production.

TABLE 6
Glucose utilization and ethanol production at different time intervals. The values for
glucose utilization and ethanol production are expressed as mg/gram of the
fermentation medium.

| | Time (h) | | | | | | | | | |
|---|---|---|---|---|---|---|---|---|---|---|
| | 0 | | 4 | | 8 | | 12 | | 24 | |
| Treatments | glucose | ethanol | glucose | ethanol | glucose | ethanol | glucose | ethanol | Glucose | Ethanol |
| Control | 685.53 | 0 | 666.27 | 17.39 | 411.75 | 159.43 | 101.68 | 317.09 | 3.24 | 356.47 |
| YE 1.0% | 700.11 | 0 | 672.77 | 19.15 | 152.16 | 292.56 | 3.61 | 370.52 | 2.98 | 345.36 |
| YE 1.5% | 708.89 | 0.00 | 668.33 | 20.53 | 152.26 | 297.49 | 4.43 | 370.51 | 4.60 | 345.28 |
| YE 2.0% | 708.79 | 0.00 | 675.57 | 20.84 | 151.86 | 301.49 | 9.06 | 371.10 | 4.72 | 345.89 |
| RB 1.0% | 713.77 | 0.00 | 674.95 | 20.86 | 362.87 | 198.36 | 7.75 | 369.16 | 2.95 | 357.10 |
| RB 1.5% | 706.60 | 0.00 | 667.19 | 20.81 | 296.33 | 223.24 | 8.35 | 369.93 | 3.45 | 362.27 |
| RB 2.0% | 691.60 | 0.00 | 685.95 | 19.29 | 274.28 | 242.21 | 9.42 | 367.25 | 3.48 | 359.71 |
| DRB 1.0% | 705.97 | 0.00 | 691.69 | 21.25 | 361.89 | 200.64 | 7.23 | 365.48 | 2.20 | 355.46 |
| DRB 1.5% | 711.12 | 0.00 | 683.51 | 21.01 | 355.92 | 209.66 | 7.94 | 367.31 | 2.36 | 355.33 |
| DRB 2.0% | 711.73 | 0.00 | 691.59 | 21.20 | 354.41 | 208.55 | 9.14 | 367.58 | 2.40 | 363.94 |

The control sample illustrated ethanol production without the addition of any of the three nutrients. In an earlier experiment, ethanol production from corn derived glucose was undertaken without the addition of antibiotics to control contamination of yeast culture. As a result, ethanol yield was reduced. The experiment was repeated and the results shown above were obtained when antibiotic lactrol at 2 PPM concentration was added prior to ethanol fermentation.

From the Table 6, it is evident that addition of exogenous nutrients allows faster ethanol production kinetics compared to the control experiment before 24 hours. Thus the presence of a exogenous nitrogen source in the fermentation medium accelerated the rate of ethanol production. Of the three exogenous nitrogen sources tested in this example, the RB and DRB are definitely much cheaper than the YE. Therefore the RB and DRB can be used as an accelerant in alcohol fermentation with out any significant increase in the cost of alcohol production.

Both RB and DRB are excellent nutrient source and can effectively replace typical nutrient sources, such as yeast extract, in the fermentation medium. Particularly, in synthetic medium containing glucose as illustrated in FIGS. 1-4, both RB and DRB yielded significantly higher ethanol concentration compared to YE. This suggests that in those bioprocesses using synthetic glucose to produce high purity products, such as biopharmaceuticals and specialty industry chemicals, RB or DRB can serve as effective nutrient source to alleviate production cost.

What is claimed is:

1. A method of producing fermentation-based products comprising the steps of:
(a) providing a fermentation medium;
(b) providing a carbohydrate substrate;
(c) adding a microorganism capable of fermenting the carbohydrate substrate;
(d) providing a bran material; and
(e) incubating the combination for a time sufficient to produce a fermentation-based product.

2. The method of claim 1, wherein the carbohydrate source is a starch derived from a plant source.

3. The method of claim 1, wherein the carbohydrate source is a lignocellulose derived from a plant source.

4. The method of claim 1, wherein the bran material is derived from a cereal grain.

5. The method of claim 1, wherein in the bran material is stabilized bran.

6. The method of claim 1, wherein the bran material is defatted bran.

7. The method of claim 1, wherein the bran material is rice bran.

8. The method of claim 1, wherein the microorganism is Saccharomyces cerevisiae.

9. The method of claim 1, wherein the microorganism is a bacteria.

10. The method of claim 1, wherein the fermentation based product is an ethanol.

11. The method of claim 1, wherein the fermentation based product is an organic acid.

12. A method for the accelerated production of a fermentation-based product, the method comprising:
(a) providing a fermentation medium;
(b) providing bran material as a source of nitrogen;
(c) adding microorganism capable of fermentation to the fermentation medium; and
(d) incubating the components for a time sufficient to produce a fermentation-based product.

13. The method of claim 12, wherein the bran material is derived from a cereal grain.

14. The method of claim 12, wherein the bran material is stabilized bran.

15. The method of claim 12, wherein the bran material is defatted bran.

16. The method of claim 12, wherein the bran material is stabilized rice bran.

17. The method of claim 12, wherein the microorganism capable of fermentation is Saccharomyces cerevisiae.

18. The method of claim 12, wherein the microorganism capable of fermentation is a bacteria.

19. The method of claim 12, wherein the fermentation-based product is an alcohol.

20. The method of claim 12, wherein the fermentation-based product is an organic acid.

21. A medium for use in accelerating the growth of a microorganism capable of fermentation, the medium comprising a bran material as a nutrient source.
